(19) United States
(12) Patent Application Publication
(10) Pub. No.: US 2025/0381112 A1
(43) Pub. Date: Dec. 18, 2025

(54) EMULSION WITH GLYCERYL CAPRYLATE AND AT LEAST ONE EMOLLIENT CHOSEN FROM MONO, DI AND/OR TRIESTER

(71) Applicant: BEIERSDORF AG, Hamburg (DE)

(72) Inventors: Shuqin XU, Wuhan (CN); Fasong LI, Wuhan (CN)

(21) Appl. No.: 18/867,521

(22) PCT Filed: May 2, 2023
(86) PCT No.: PCT/EP2023/061468

§ 371 Date: Nov. 20, 2024

(30) Foreign Application Priority Data
Jun. 1, 2022 (WO) PCT/CN2022/096614

(51) Int. Cl. A61K 8/37; A61K 8/06; A61Q 19/00

(52) U.S. Cl. CPC A61K 8/375; A61K 8/062; A61Q 19/00; A61K 2800/30; A61K 2800/34; A61K 2800/48

(57) ABSTRACT

Disclosed is a cosmetic skincare emulsion being free from silicones, synthetic polymers, polyalkoxylated compounds especially polyethylene glycols and polyethylene derivatives. Upon distribution on the skin the emulsion develops hardly any foam and in consequence do not leave any whitish residues on the skin. The emulsions according to the invention absorb well into the skin.

### TECHNICAL FIELD

The present invention belongs to the cosmetic field, and relates to cosmetic skincare emulsions, which are very suitable for cosmetic face care and body care. The emulsions are free from silicones, synthetic polymers, polyalkoxylated compounds, especially polyethylene glycols and polyethylene derivatives. Upon distribution on the skin the emulsions develop hardly any foam and in consequence do not leave any whitish residues on the skin. The emulsions according to the invention absorb well into the skin.

### BACKGROUND ART

The skin is the largest human organ and has many functions (for example, the organ of perception and temperature regulation). However, the most important function is the barrier function, which prevents the skin, and ultimately the entire organism, from drying out. At the same time, the skin protects from penetration and absorption of external substances, which may be toxic or otherwise harmful.

The skin is composed of layers, namely the epidermis, a basement membrane, the dermis, and the subcutaneous tissue. The epidermis is the outmost layer consisting of Stratum Corneum, Stratum Lucidum, Stratum Granulosum, Stratum Spinosum and Stratum Basale. The corneocytes are the main cell type of the epidermis.

The Stratum Corneum (horny layer) is essential in establishing the barrier function of the skin. The “Elias” skin model, which is widely accepted in the field of dermatology and cosmetic (P. M. Elias, Structure and Function of the Stratum Corneum Permeability Barrier, Drug Dev. Res. 13, 1988, 97-105), describes the horny layer as a two-component system, similar to a brick wall (bricks and mortar model). In this model, the horny cells (corneocytes) correspond to the bricks, and the lipids in the intercellular spaces, which are of a complex composition, correspond to the mortar. This system is essentially a physical barrier to hydrophilic substances, but, because of its narrow and multilayered structure, also lipophilic substances can pass only to a limited extent.

Further structures contribute to the stability and function of the stratum corneum, hook-like structures of the corneocytes, corneo desmosomes, the bilayer of the stratum corneum and tight junctions.

Epidermal lipids contribute to the integrity of the horny layer. Epidermal lipids also affect the smoothness of the skin. In contrast to lipids secreted by the sebaceous glands, which do not form a continuous film on the skin, the epidermal lipids are evenly distributed over the entire horny layer.

The extremely complex interaction of moisture-binding substances and lipids in the upper layers of the skin is very important for the regulation of skin moisture. For this reason, cosmetics generally comprise, in addition to balanced lipid mixtures and water, water-binding substances.

The aim of cosmetic skin care is primarily to support the natural function of the skin as a barrier against environmental influences (e.g. dirt, chemicals, microorganisms) and against the loss of endogenous substances (e.g. water, natural fats, electrolytes). Cosmetic skin care shall strengthen and/or contribute to restore the natural condition of the skin.

If the barrier function is impaired, toxic or allergenic substances may penetrate or microorganisms may infiltrate the skin, leading to toxic, allergic, or inflammatory reactions.

Another aim of skin care is to replenish lipids and water, which are lost by the daily washing routine. This becomes particularly important in cases when the natural regeneration ability of the skin is impaired. Furthermore, skincare products should protect against environmental influences, in particular against sun and wind, and help to delay skin aging.

Frequently, skin care products are made available in form of an emulsion. Generally, emulsions are understood as a heterogeneous system of two liquids which are immiscible or miscible only to a limited extent with one another and usually referred to as phases. One liquid is present in form of droplets (disperse or internal phase), whilst the other one forms a continuous (coherent or internal) phase. The liquids (pure or as solutions) are present in an emulsion in a more or less fine distribution, which is generally of only limited stability.

If the two liquids are water and oil and oil droplets are finely dispersed in water, this composition is an oil-in-water emulsion (O/W emulsion, e.g. milk). The basic properties of an O/W emulsion are determined by the water phase, the basic properties being for example electrical conductivity, sensory properties, ability of the continuous phase to be stained.

In the case of a water-in-oil emulsion (W/O emulsion, e.g. butter), where droplets of water or an aqueous phase are finely dispersed in the oil phase, the basic properties are determined by the oil or oily phase.

In order to be able to ensure the stability of emulsions, interface-active substances, i.e. emulsifiers, are usually necessary. Additionally to the necessary emulsifier(s) the stability of emulsions may be improved by the incorporation of synthetic polymers, frequently based on acrylic acid and/or derivatives thereof.

As the consumer becomes more and more aware of the aspect that the products, they use should be eco-friendly, there is a need to provide products complying with this desire. The cosmetic products shall be mainly composed of raw materials which are of natural origin or at least nature-based and readily biodegradable.

The term biodegradability describes the process of the breakdown of organic compounds by living organisms, especially saprobionts. Ideally, inorganic substances such as CO₂, O₂ and ammonia are produced, compounds that are used by plants and microorganisms to build up organic compounds again.

Organic, chemical compounds that are readily biodegradable are classified according to OECD 301, for cosmetic compositions mostly OECD 301 B. These substances can be broken down quickly and completely.

Organic, chemical compounds that are classified according to OECD 302 are restricted, but basically biodegradable.

The term “natural” in connection with natural ingredients for cosmetics is specified in ISO 16128; the first part of ISO 16128-1 was published on Feb. 15, 2016, the second part of ISO 16128-2 was published in September 2017. According to this guideline, natural, cosmetic ingredients can be summarized as substances that are available from plants, animals, minerals or microorganisms, including those substances that are obtained from the above-mentioned sources by physical processes, fermentation processes (only those fermentation processes that are also used to occur in nature and lead to products that are also obtained in nature), and other manufacturing methods without chemical modification. The microorganisms may only be those that have not been genetically modified.

In general, nature-based substances are compounds that are derived from natural starting materials or are produced starting from natural starting materials. In order to assess the extent to which a substance is nature-based, the method for determining the “Biorenewable Carbon Index” (=BCl) may be used. This method is used to determine the percentage of carbon atoms that come from renewable plant or animal sources in relation to the carbon atoms from non-renewable, petrochemical sources, see HAPPI Jul. 58-60, 2009.

Hence, the problem of the present invention was to provide emulsions, especially O/W emulsions, which mainly comprise raw materials, which are of natural origin or naturally derived.

Cosmetic compositions, which are mainly composed of natural, or nature based raw materials, are known and already available on the market. Frequently, such products are made available by companies which distribute so called natural cosmetics. Products of the company of Weleda are examples for such products. Exemplary, the products “Calendula Pflegemilch” (Mintel data base, record number 8405473), “Express-Feuchtigkeit Körperlotion Citrus” (Mintel data base, record number 8991140), and “Feigenkaktus 24 h Feuchtigkeitscreme (Mintel data base, record number 8060985) may be mentioned.

However, the person skilled in the art, developing products being mainly composed of raw materials of natural origin or raw materials being naturally derived is up against the problem to substitute well-known, well established raw materials such as silicones and/or synthetic polymers, e.g. polymers based on acrylic acid and/or derivatives thereof, polyalkoxylated compounds, especially polyethylene glycols and polyethylene derivatives. The replacement of the silicone oils by raw materials providing similar sensory and caring properties to the compositions resulted in compositions, which developing a foam which is not absorbed into the skin in a quick and easy way but left a film on the skin which was whitish and visible for considerable time.

In the cosmetic industry raw materials characterized by the presence of polyalkoxylated moieties, especially polyethylene glycol moieties in the molecule are frequently used. This holds true for example for emulsifiers or emulsifying substances. Polyethylene glycol is often abbreviated to PEG, and the polyethylene glycol moieties are often referred to as PEGs for short. Polyethylene glycol moieties consist of at least two ethylene glycol molecules linked to each other, there can also be hundreds of ethylene glycol molecules, for example 200. Ingredients containing these polyethylene glycol moieties are increasingly undesirable to consumers. It was thus also the task of the present invention not to incorporate ingredients containing polyethylene glycol moieties into the emulsions according to the invention.

The emulsions according to the invention do not contain any silicones or silicone-containing compounds, which means that no silicones and/or silicone-containing compounds and no raw materials which contain silicones or silicone-containing compounds, even in traces, are added to the emulsions of the present invention. Silicone-containing compounds are characterized by the presence of silicon and oxygen atoms, which are linked to one another and can form a large number of different compounds.

Polymers are high-molecular chemical compounds (macromolecules) made up of repeating units (monomers), which can be the same or different. Polymers can have linear, branched, or crosslinked structures. Polymers can be classified as synthetic polymers if they are formed by synthetic reactions. A frequently used monomer is acrylic acid or derivatives thereof. Synthetic polymers have no equivalent in nature and are not biodegradable in usual time intervals. The consequence is that these polymers persist in nature for a very long time contributing to the pollution of the landscape and waters.

### SUMMARY OF THE INVENTION

Hence, the problem of the present invention was to provide an emulsion, especially an O/W emulsion containing mainly raw materials which were of natural origin or naturally derived, which were well and easily absorbed into the skin without developing a foam which may become visible as a whitish film on the skin.

Surprisingly, the problem was solved by a cosmetic emulsion, preferably an O/W emulsion containing
glyceryl caprylate and
at least one emollient chosen from neopentenyl glycol diheptanoate, isodecyl neopentanoate, triheptanoin, diisopropyl adipate, and/or squalane.

Glyceryl caprylate is a monoester of glycerol with caprylic acid. Glyceryl caprylate is an advantageous component for cosmetic compositions because it has multiple functions. Due to its chemical nature glyceryl caprylate has emulsifying properties. It also regulates moisture and moisturizes the skin. In addition, it is extraordinarily skin-compatible. As glyceryl caprylate is active against bacteria and yeasts it has a stabilizing effect for cosmetic compositions.

Glyceryl caprylate may be purchased from the company Evonik Operations GmbH under the trade name Dermosoft GMCY MB.

In the emulsion according to the invention glyceryl caprylate is contained in an amount of 0.05 to 3.0% by weight, preferably 0.1 to 0.5% by weight in relation to the total weight of the emulsion. The percent values are relating to the active content values.

In addition to glyceryl caprylate one or more further emulsifier(s) or component(s) with emulsifying properties may be contained, which may preferably be chosen from glyceryl stearate, polyglyceryl-3 methyl glucose distearate, and/or Glyceryl Stearate Citrate

If in the emulsion according to the invention the at least one further emulsifier or component with emulsifying properties is contained, said emulsifier is contained in a total amount of 0.5 to 5.0% by weight, preferably 1.0 to 4.5% by weight, in relation to the total weight of the emulsion. The percent values are relating to the active content values.

Due to the chemical nature of the above mentioned emulsifiers containing at least one fatty acid residue, which is of hydrophobic nature, the emulsifiers may also be effective as emollients, at least to some extent.

In the emulsion according to the invention at least one emollient chosen from neopentenyl glycol diheptanoate, isodecyl neopentanoate, triheptanoin, diisopropyl adipate, and/or squalane is contained.

The emollients in form of esters which are described below are at least naturally derived because all the alcohols and the fatty/carboxylic acids are obtained from natural sources.

Diisopropyl adipate may be purchased from the company Symrise AG under the trade name Isoadipate 660014.

Neopentyl glycol diheptanoate may be purchased from Inolex Chemical Company under the trade name Lexfeel 7.

Isodecyl neopentanoate may be purchased from the company the company Stéarinerie Dubois Fils under the tradename DUB VCI 10.

Triheptanoin may be purchased from the company of Evonik Operations GmbH under the trade name DERMOFEEL TC-7.

The emollient may be squalene, having 30 carbon atoms. Squalane is an acyclic hydrocarbon, a basic building block of triterpenes. Squalane may be sourced from the livers of sharks, olive oil, rice, and sugar cane. Moreover, hemisqualane, having 15 carbon atoms, may also be used. In comparison to squalane, hemisqualane is not as effective as squalane in improving the absorption of the emulsion on the skin.

Squalane may be purchased from the company of EFPBiotek under the trade name Olive Squalane.

The incorporation of one or more of the above mentioned emollients results in an emulsion which develops hardly any foam. Hence, the emulsions absorbs into the skin nearly without any whitish residues. If foam develops it is only to a small extent. The foam absorbs well and there is hardly any whitish residue detectable.

In the emulsion according to the present invention the at least one emollient chosen from neopentenyl glycol diheptanoate, isodecyl neopentanoate, triheptanoin, diisopropyl adipate, and/or squalane is contained in a total amount of 0.5 to 10% by weight, more preferably 1.0 to 5.0% by weight, referring to the total weight of the emulsion. The values are referring to the active content of the respective emollient.

Additionally to the above mentioned emollients at least one further emollient may be contained in the emulsion according to the invention.

The at least one further emollient may be chosen from at least one triglyceride with the proviso that triheptanoin is not comprised. The fatty acids of the triglyceride are chosen from a mixture of fatty acids, as they are derived from natural sources. Esters of glycerine with fatty acids obtained from natural oils such as coconut oil, palm oil, soybean oil and others are advantageous. Such fatty acids are in each case mixtures that reflect the composition of the respective natural oils. Coco glycerides is a particularly advantageous example. Coco glycerides may be purchased from the company of BASF under the trade name Myritol 331.

The at least one further emollient may be chosen from esters of isopropyl alcohol and fatty acids having 6 to 22 carbon atoms, preferably 8 to 20 carbon atoms. Isopropyl myristate, isopropyl palmitate, and isopropyl stearate are advantageous examples, isopropyl palmitate being particularly advantageous.

Myristyl myristate is also an advantageous emollient.

In the emulsion according to the invention combinations of the above mentioned emollients may be contained.

Additionally, in the emulsion according to the invention esters different from those as described above may also be contained, for example isoamyl laurate and/or dialkyl ether, such as dicaprylyl ether.

Due to their chemical nature, namely due to an amphiphilic nature, the emollients in form of esters may have emulsifying properties, at least to a certain extent.

If in the emulsion according to the present invention at least one further emollient is contained this further emollient is contained in a total amount of 3.0 to 25% by weight, preferably 5.0 to 20% by weight, referring to the total weight of the emulsion. The values are referring to the active content of the respective emollient.

In the emulsion according to the invention at least one thickener may be contained, which is of natural origin or naturally derived.

In general, thickeners are macromolecules, which have a mainly linear configuration and also have varying degree intermolecular forces of interaction permitting secondary and primary valence bonds between individual residues of the molecule and thus the formation of a reticular structure. Thickeners may be water-soluble or water-swellable, natural or synthetic polymers, which form gels or viscous solutions in aqueous systems. They increase the viscosity of water by either binding water molecules (hydration) or else by absorbing and encapsulating the water into their macromolecules, which may be interwoven; both effects result in restricting the mobility of the water. Such water-soluble or water-swellable polymers represent a large group of chemically very different natural and synthetic polymers, whose common feature is their solubility or swellability in water or aqueous media. A precondition for the solubility or swellability in water is a sufficient high number of hydrophilic groups for ensuring the solubility in water and a limited crosslinking. The hydrophilic groups may be nonionic, anionic, or cationic groups.

According to the invention, the thickeners are chosen from the following groups:
organic, natural compounds, such as, for example, agar agar, carrageen, tragacanth, gum arabic, alginates, pectins, polyoses, guar flour, carob bean flour, starch, dextrins, gelatin, casein,
organic, modified natural substances, such as, for example carboxymethylcellulose and other cellulose ethers, hydroxyethylcellulose and hydroxypropylcellulose and the like.

Advantageously, the thickeners are biopolymers from various sources, more advantageously, the at least one thickener is selected from the group of xanthan gum, gellan gum, succinoglycan gum, scleroglucan, celluloses and/or derivatized celluloses; most advantageously, the at least one thickener is chosen from xanthan gum, gellan gum and/or hydroxypropyl starch phoshate.

For instance, Xanthan gum, an exopolysaccharide, is obtained from Xanthomonas campestris. Xanthan gum is a polymer that consists of a main chain of β-1,4 glycosidically linked glucose molecules. Every second glucose residue has a side chain made up of two mannose residues, one glucuronic acid residue and pyruvate.

Xanthan gum dissolves well in hot and cold water, forming single and double helices. A three-dimensional network is created.

Xanthan gum can be obtained, for example, under the trade name Keltrol CG-RD from CP Kelco.

Gellan gum is an unbranched anionic polymer; the basic unit of this polymer is a tetrasaccharide of two glucose residues, glucuronic acid and rhamnose. Nearly every tetrasaccharide is esterified with glycerate and nearly every second basic unit is esterified with acetate. Both substituents are attached to the same glucose residue. Native gellan gum is highly acetylated. It forms soft, elastic, and non-brittle gels. The gels of gellan gum, which is acetylated to a lower degree are firm, non-elastic and brittle.

Gellan gum advantageous according to the invention can be purchased, for example, from the company CP Kelco under the trade name Kelcogel CG-LH.

Hydroxypropyl starch phosphate, which is a naturally derived starch, may be obtained by esterification of food starch with sodium trimetaphosphate or phosphorus oxychloride, combined with etherification by propylene oxide. Hydroxypropylation results in the substitution of hydroxyl groups with 2-hydroxypropyl ether.

Hydroxypropyl starch phosphate may be cold processed, which is of great advantage.

Hydroxypropyl starch phosphate may be purchased from the company of Cargill under the trade name Stardesign Care.

If in the emulsion according to the present invention the at least one thickener is contained this thickener is preferably contained in a total amount of 0.05 to 5.0% by weight, more preferably 0.1 to 3.5% by weight, referring to the total weight of the emulsion. The values are referring to the active content of the thickener.

The incorporation of octocrylene has positive effects. On one hand it became apparent that hardly any foam develops on the skin upon distributing the inventive emulsion containing octocrylene. Hence, only very little whitish residues, if at all, become apparent. On the other hand octocrylene is a known UV-filter providing a UV-screen mainly against UV-B rays.

If octocrylene is contained in the emulsion according to the present invention, it is contained in an amount of 0.5 to 8.0% by weight, preferably 1.0 to 5.0% by weight, referring to the total weight of the emulsion.

Further UV-filters may be contained in the emulsions according to the present invention. The UV-filters may be chosen from titanium dioxide and/or zinc oxide, both of which may be coated; triacine, butyl methoxydibenzolmethane, diethylamino hydroxybenzoyl hexyl benzoate, bis-ethylhexyloxyphenol methoxyphenyl triazine.

If at least one further UV-filter is contained in the emulsion of the present invention, the at least one further UV-filter is contained in a total amount of 1.0 to 10% by weight, preferably 1.0 to 5.0% by weight, referring to the total weight of the emulsion.

The emulsion of the present invention is an aqueous composition. Preferably, water is contained in an amount of 60 to 90% by weight, more preferably in an amount of 65 to 80% by weight, referring to the total weight of the emulsion.

Advantageously, according to the invention additionally at least one moisturizer is contained in the emulsion of the present invention. Moisturizers are hygroscopic substances, which bind water and thereby provide moisture. The hygroscopic quality is due to hydrophilic substituents of the molecule, in many cases hydroxyl groups, but other functional groups as amine or carboxyl groups have the same effect. Examples of humectants are propylene glycol, butylene glycol, caprylyl glycol, glycerine, sorbitol, xylitol, maltitol, polydextrose, hydrogenated starch hydrolysate, urea, Aloe vera gel, alpha hydroxy acids such as lactic acid, honey. Preferred moisturizers are glycerine and/or caprylyl glycol, which are obtained from natural processes.

If at least one moisturizer is contained in the emulsion of the present invention, the at least one moisturizer is preferably contained in a total amount of 1.0 to 10.0% by weight, more preferably 2.0 to 8.0% by weight, referring to the total weight of the emulsion.

Advantageously, according to the invention the emulsion shall be stable in relation to microbial contamination. In general, in order to reduce the number of microorganisms and prevent the growth of microorganisms preservatives are added to cosmetic compositions. However, many of the known preservatives are of synthetic origin, hence not natural.

In one embodiment an emulsion is provided, which does not contain any preservative of synthetic origin. In this case the emulsion may be preserved by adding short chain alcohols, preferably chosen from ethanol and/or isopropanol, which are obtained from natural processes such as fermentation.

In a further embodiment an emulsion is provided, which contains at least one preservative, which is according to the list of approved preservatives of the European Union (EC regulation 1223/2009; Annex V). It is preferred if the preservative is phenoxyethanol.

If phenoxyethanol is contained in the emulsion according to the present invention phenoxyethanol is contained in an amount of 0.1 to 9.0% by weight, preferably, 0.4 to 0.8% by weight, referring to the total weight of the emulsion.

The emulsion of the present invention is preferably an O/W-emulsion. It is preferred that the oil phase is contained in an amount of 3 to 25% by weight, more preferably in an amount of 5 to 20% by weight. The amounts in % by weight are referring to the total weight of the emulsion.

The emulsion of the present invention is free from silicones, synthetic polymers, polyalkoxylated compounds, especially polyethylene glycols and polyethylene derivatives.

The composition of the present invention may be prepared by any technique known or effective to prepare an O/W emulsion or a W/O emulsion. The process to prepare the composition of the present invention comprises conventional formulating and mixing techniques.

In order to evaluate the emulsions of the present invention in relation to the development of a foam and the leaving of a whitish residue on the skin a method was developed to analyze whitish residues on the skin using an image analyzer and an image analyze software (ImageJ).

The following steps were conducted:
Set up instruments:
   1. Prepare samples and filling them into a pipette.
   2. Set a metronome (96 beep per minute).
   3. Prepare a stopwatch.
   4. Turn on the light box and adjust to white light.
   5. Prepare the artificial skin, which is of artificial leather, and reference cards.
   6. Turn on the camera and set the camera.
Apply the sample on the artificial skin and take picture:
   1. Apply 200 μL of the emulsion on the left third of the artificial skin.
   2. Start the metronome.
   3. Start the stopwatch, spread the product on the artificial skin by moving 3 fingers right and left to the beat of the metronome for 30 sec.
   4. Take the picture immediately after the finish of application.
Analyses the picture(s) using ImageJ (software):
Result calculation:
Set the relative anti-whitish value of standard white area (2*10 cm) as 0%;
Set the relative anti-whitish value of total black (L-value<50, mean gray value=255) as 100%.

$$Relative anti whitish ( \% ) = \frac{{M}_{sample} - {M}_{20}}{255 - {M}_{20}} \times 100 \%$$

Mₛₐₘₚₗₑ: Mean gray value of test sample.
M₂₀: Mean gray value of 2×10 cm standard white area.

### Technical Effects

It becomes apparent that a composition according to the comparative example A containing cyclomethicone as an emollient and a carbomer as a thickener produces only very little foam. In general, said foam has a whitish appearance and does not absorb very quickly to the skin. Hence, it was the aim of the present invention to arrive at an emulsion without an acrylic thickener and without any silicone compound but nevertheless producing only little foam and hence leaving nearly no whitish residues on the skin.

The results which were obtained from the analysis showed that the replacement of the thickener carbomer by the thickeners xanthan gum and hydroxypropyl starch phosphate and the substitution of silicone by coco glycerides and isopropyl palmitate resulted in the development of a relatively prominent foam and in consequence whitish residues on the skin (comparative example B).

Starting from these data the several emulsifiers were tested. Especially by the incorporation of glyceryl caprylate improved results could be achieved.

Furthermore, different emollients were analyzed. It became apparent that several emollients, namely neopentenyl glycol diheptanoate, isodecyl neopentanoate, triheptanoin, diisopropyl adipate, and/or squalane resulted in a further reduction of the whitish residues. The whitish residues were diminished, being at least 70% the value of the comparative composition containing silicone oils and carbomer (comparative composition A).

The emulsion containing the emollient diisopropyl adipate was nearly as good as the comparative emulsion, 96.71% (best inventive emulsion, example 6) in comparison to 98.94% (comparative emulsion A).

Furthermore, an emulsion of octocrylene showed very satisfying results. The development of foam was clearly reduced and hence only very few whitish residues become apparent. This effect was shown by the relative anti-whitish value of 79.74% for the composition according to example.

The results of the analysis of the example compositions shown in table 2 further support the finding that the use of glyceryl caprylate and diisopropyl adipate result in compositions nearly as good as the compositions containing carbomers and silicones.

It is shown that the emulsions according to the invention solve the problem of the present invention.

### EXAMPLES

The examples below are intended to illustrate the present invention without limiting it. The numerical values in the examples are percentages by weight, based on the total weight of the particular composition. The values refer to active content.

TABLE 1

| | example | | | | | | | |
|---|---|---|---|---|---|---|---|---|
| | compar- | compar- | | | | | | |
| INCI names | ative A | ative B | inventive 1 | inventive 2 | inventive 3 | inventive 4 | inventive 5 | inventive 6 |
| Ethylhexyl Cocoate | 2 | | | | | | | |
| Cyclomethicone | 5 | | 0 | 0 | 0 | 0 | 0 | 0 |
| Cocoglycerides | | 4 | 4 | 4 | 4 | 4 | 4 | 4 |
| Isopropyl Palmitate | | 2 | 2 | 2 | 2 | 2 | 2 | 2 |
| C12-15 Alkyl Benzoate | 2 | 2 | 0 | 0 | 0 | 0 | 0 | 0 |
| Neopentyl Glycol Diheptanoate | | | 2 | 0 | 0 | 0 | 0 | 0 |
| Squalane | | | 0 | 2 | 0 | 0 | 0 | 0 |
| Octocrylene | | | 0 | 0 | 2 | 0 | 0 | 0 |
| Isodecyl Neopentanoate | | | 0 | 0 | 0 | 2 | 0 | 0 |
| Triheptanoin | | | 0 | 0 | 0 | 0 | 1.2 | 0 |
| C13-16 Isoalkane | | | | | | | 0.8 | |
| Diisopropyl Adipate | | | | | | | | 2 |
| Polyglyceryl-3 | 3.25 | 3.25 | 3.25 | 3.25 | 3.25 | 3.25 | 3.25 | 3.25 |
| Methylglucose Distearate | | | | | | | | |
| Glyceryl Stearate | | 1 | 1 | 1 | 1 | 1 | 1 | 1 |
| Glyceryl Caprylate | | | 0.2 | 0.2 | 0.2 | 0.2 | 0.2 | 0.2 |
| Tapioca Starch | 3.4 | 3.4 | 3.4 | 0 | 0 | 0 | 0 | 0 |
| Parfum | 0.25 | 0.2 | 0 | 0 | 0 | 0 | 0 | 0 |
| Glycerin | 7.5 | 7.5 | 7.5 | 7.5 | 7.5 | 7.5 | 7.5 | 7.5 |
| Methylpropanediol | 2 | | 0 | 0 | 0 | 0 | 0 | 0 |
| Caprylyl Glycol | | 0.35 | 0.35 | 0.35 | 0.35 | 0.35 | 0.35 | 0.35 |
| Sodium Hydroxide | 0.12 | | | | | | | |
| Citric Acid | | 0.2 | 0.2 | | | | | |
| Phenoxyethanol + Aqua | 0.6 | 0.8 | 0.8 | 0.8 | 0.8 | 0.8 | 0.8 | 0.8 |
| Methylparaben | 0.3 | | | | | | | |
| Cetearyl Alcohol | 1.25 | 1.25 | 1.25 | 1.25 | 1.25 | 1.25 | 1.25 | 1.25 |
| Carbomer | 0.3 | | | | | | | |
| Xanthan Gum | | 0.2 | 0.2 | 0.2 | 0.2 | 0.2 | 0.2 | 0.2 |
| Hydroxypropyl Starch Phosphate | | 1.5 | 1.5 | 1.5 | 1.5 | 1.5 | 1.5 | 1.5 |
| Aqua | 71.34 | 71.75 | 71.75 | 75.95 | 75.95 | 75.95 | 75.95 | 75.95 |
| Anti-Whitish Foaming | 98.94% | 39.47% | 70.36% | 72.94% | 79.74% | 82.79% | 85.92% | 96.71% |
| (Image Analyze, % values) | | | | | | | | |

TABLE 2

| | example | | | | | | | |
|---|---|---|---|---|---|---|---|---|
| | comparative | comparative | comparative | inventive | inventive | inventive | inventive | inventive |
| INCI names | C | D | E | 7 | 8 | 9 | 10 | 11 |
| Myristyl Myristate | 1 | 1 | 1 | 1 | 1 | 1 | 1 | 1 |
| Hydrogenated Coco-Glycerides | 3 | 3 | 3 | 3 | 3 | 3 | 3 | 3 |
| Dimethicone | 0.9 | | | | | | | |
| Ethylhexyl Cocoate | 4 | 3 | 3 | 3 | | | | |
| Isopropyl Palmitate | 3 | 3 | | | | | | |
| Dicaprylyl Ether | | | | | 3 | 3 | 3 | 3 |
| Diisopropyl Adipate | | | 3 | 3 | 3 | 3 | 3 | 3 |
| Glyceryl Caprylate | | | | 0.2 | 0.2 | 0.2 | 0.2 | 0.2 |
| Glyceryl Stearate Citrate | 2 | 2 | 2 | 2 | 2.5 | 2 | 2.2 | 2 |
| Distarch Phosphate | | 2 | 2 | 2 | 2 | 2 | 2 | 2 |
| Aluminum Starch Octenylsuccinate | 2 | | | | | | | |
| Parfum | 0.3 | 0.3 | | | | | | |
| Glycerin | 8 | 7.5 | 7.5 | 7.5 | 7.5 | 7.5 | 7.5 | 7.5 |
| 1,2-Hexanediol | 0.75 | 0.75 | 0.75 | 0.75 | 0.75 | 0.75 | 0.75 | 0.75 |
| Methylpropanediol | 2 | 2 | 2 | 2 | 2 | 2 | 2 | 2 |
| Sodium Hydroxide | 0.13 | | | | | | | |
| Phenoxyethanol | 0.6 | 0.6 | 0.8 | 0.8 | 0.6 | 0.6 | 0.6 | 0.8 |
| Benzyl Alcohol | 0.3 | 0.3 | 0.3 | 0.3 | 0.3 | 0.3 | 0.3 | 0.3 |
| Trisodium EDTA | 0.1 | | | | | | | |
| Cetyl Alcohol | 3 | 3 | 4 | 4 | 3 | 3 | 3 | 3 |
| Hydroxypropyl Starch Phosphate | | 1.5 | 1.5 | 1.5 | 1.5 | 1.5 | 1.5 | 1.5 |
| Xanthan Gum | | 0.3 | 0.3 | 0.3 | 0.3 | 0.3 | 0.3 | 0.3 |
| Carbomer | 0.5 | | | | | | | |
| Calcium Chloride | 0.01 | | | | | | | |
| Sodium Chloride | 0.01 | | | | | | | |
| Aqua | 67.75 | 69.03 | 68.85 | 68.65 | 69.3 | 69.5 | 69.6 | 69.55 |
| Anti-Whitish Foaming | 99.15% | 81.72% | 85.85% | 91.73% | 94.40% | 92.23% | 93.46% | 94.08% |
| (Image Analyze, % values) | | | | | | | | |

1.-18. (canceled)

19. A cosmetic emulsion, wherein the emulsion comprises glyceryl caprylate and (i) at least one emollient selected from neopentenyl glycol diheptanoate, isodecyl neopentanoate, triheptanoin, diisopropyl adipate, and squalane and/or (ii) octocrylene.

20. The emulsion of claim 19, wherein the emulsion is an O/W emulsion.

21. The emulsion of claim 19, wherein at least (i) is present.

22. The emulsion of claim 19, wherein at least (ii) is present.

23. The emulsion of claim 19, wherein (i) and (ii) are present.

24. The emulsion of claim 19, wherein the emulsion comprises from 0.05% to 3.0% by weight of glyceryl caprylate, based on a total weight of the emulsion.

25. The emulsion of claim 19, wherein the emulsion comprises from 0.5% to 10% by weight of (i), based on a total weight of the emulsion.

26. The emulsion of claim 19, wherein the emulsion comprises from 0.5% to 8% by weight of (ii), based on a total weight of the emulsion.

27. The emulsion of claim 19, wherein the emulsion further comprises at least one emulsifier selected from glyceryl stearate, polyglyceryl-3 methyl glucose distearate, and Glyceryl Stearate Citrate.

28. The emulsion of claim 27, wherein the emulsion comprises the at least one emulsifier in a total concentration of from 0.5% to 5.0% by weight, based on a total weight of the emulsion.

29. The emulsion of claim 19, wherein the emulsion further comprises at least one emollient selected from coco glycerides, isopropyl myristate, isopropyl palmitate, isopropyl stearate, and myristyl myristate.

30. The emulsion of claim 29, wherein the emulsion comprises the at least one emollient in a total concentration of from 3.0% to 25% by weight, based on a total weight of the emulsion.

31. The emulsion of claim 19, wherein the emulsion further comprises at least one thickener.

32. The emulsion of claim 31, wherein at least one thickener is selected from one or more of xanthan gum, gellan gum, succinoglycan gum, scleroglucan, celluloses, and derivatized celluloses.

33. The emulsion of claim 31, wherein the emulsion comprises the at least one thickener in a total concentration of from 0.05% to 5% by weight, based on a total weight of the emulsion.

34. The emulsion of claim 19, wherein the emulsion comprises from 60% to 90% by weight of water, based on a total weight of the emulsion.

35. The emulsion of claim 19, wherein the emulsion further comprises at least one UV filter selected from optionally coated titanium dioxide and/or zinc oxide, triacine, butyl methoxydibenzolmethane, diethylamino hydroxybenzoyl hexyl benzoate, and bis-ethylhexyloxyphenol methoxyphenyl triazine.

36. The emulsion of claim 35, wherein the emulsion comprises the at least one UV filter in a total concentration of from 1.0% to 10% by weight, based on a total weight of the emulsion.

37. The emulsion of claim 19, wherein the emulsion is free from silicones, synthetic polymers, polyalkoxylated compounds, polyethylene glycols and polyethylene derivatives.

38. A method of reducing the development of foam and/or diminishing whitish residue on skin of a cosmetic emulsion, wherein the method comprises incorporating into the emulsion glyceryl caprylate and (i) at least one emollient selected from neopentenyl glycol diheptanoate, isodecyl neopentanoate, triheptanoin, diisopropyl adipate, and squalane and/or (ii) octocrylene
